(19) United States
(12) Patent Application Publication
(10) Pub. No.: US 2004/0015137 A1
(43) Pub. Date: Jan. 22, 2004

(54) Fluid material dispensing syringe

(75) Inventors: Ingrid Elaine Hohlfelder, Geneva, IL (US); Chester L. Zdanowski, Westmont, IL (US)

Correspondence Address: Douglas J. Hura, DENTSPLY INTERNATIONAL INC., 570 West College Avenue, York, PA, 17405-0872, US

(73) Assignee: DENTSPLY Research & Development Corp.

(21) Appl. No.: 10/618,899

(22) Filed: Jul. 14, 2003

Related U.S. Application Data
(62) Division of application No. 10/024,881, filed on Dec. 19, 2001.
(63) Continuation of application No. 09/861,380, filed on May 18, 2001, now abandoned.
(60) Provisional application No. 60/205,037, filed on May 18, 2000.

(51) Int. Cl. A61M 5/00

(52) U.S. Cl. 604/246; 604/131

(57) ABSTRACT

A dental syringe (10) has a needle (11) affixed to a carpule holder (12), which carpule holder (12) is affixed to a power drive unit (13). A harpoon (20) is provided to affix the drive unit to the carpule plunger (32). The harpoon (20) is made of stainless steel or other hard, corrosion resistant, sterilizable, material. Harpoon 20 has a unique barb geometry and hardened, knife-like, edges (21). The syringe (10) may operate to divide the delivery of anesthesia into two phases. According to the method, during the first about 10 seconds of the injection, anesthetic is delivered at an extremely slow rate to maximize patient comfort. The injection rate then automatically increases to the preprogrammed rate associated with the injection type selected.

### RELATED APPLICATION

This application is a Continuation of U.S. Ser. No. 09/861,380 filed May 18, 2001, which claims the benefit of U.S. Provisional Application Serial No. 60/205,037 filed on May 18, 2000.

### TECHNICAL FILED

The present invention is directed toward a syringe for dispensing a fluid material such as a dental anesthesia.

### BACKGROUND OF THE INVENTION

Dental anesthesia is known to be delivered from pre-filled, single use, carpules with a glass barrel and rubber-like plunger (by “rubber-like” it means actual rubber or some other material having physical properties similar to rubber). There are a variety of hand-operated dental syringes which hold these anesthesia carpules and drive the rubber plunger forward. In most clinical procedures, the dentist performs an aspiration to determine if a blood vessel has been entered, before injecting the anesthetic. (Injecting the anesthetic in the bloodstream is potentially hazardous.) Aspiration is accomplished by briefly retracting the carpule plunger to create a slight vacuum. There are a variety of means to retract the plunger, including the following: various mechanical hooks, harpoons, barbs, and corkscrews which embed in and grip the carpule plunger; a gripping member on the tip of the syringe plunger penetrates and grips the carpule's elastomeric seal. The gripping members are variously hooks, pointed needles, barbed needles, or corkscrews. This method is common in thumb-actuated syringes. One shortcoming of this method is that the carpule must be rapidly jammed onto the gripping member in order to embed it into the carpule seal without excess expulsion of anesthetic fluid. Another drawback is that various designs of gripping members either pull out of the carpule seal prematurely, or are not easily removed after injection is completed.

Sealed syringe plungers which create a vacuum behind the carpule plunger are known. A secondary seal on the syringe plunger creates a slight vacuum behind the carpule seal so the carpule seal retracts when the syringe plunger is pulled back. This method is used in a product known as The Wand computer controlled syringe. A drawback of this method is that the carpule seal is not consistently retracted. Another drawback is that the syringe plunger seal must be periodically cleaned, lubricated, or replaced.

Methods which create a vacuum in the carpule by distorting its needle septum are known. In this method, the syringe induces relative motion between the carpule and it's seal, creating the slight vacuum. In a variation of this method, the syringe induces a deflection in the carpule's septum, creating a slight vacuum in the carpule.

It has been found in laboratory tests, that none of these method work reliably, failing in one or more of the following ways: did not penetrate certain brands of carpules with high durometer rubber plungers; requiring excessive user effort or skill; pulling out of the carpule plunger and therefore failing to create an aspiration vacuum; and/or, becoming loaded with the silicone lubricant used on these carpule plungers, and then failing to grip the plunger.

A harpoon design which solves these problems is desirable.

Further, previous dental anesthesia syringes have had several problems in their operation: only a single injection rate for all procedures; only crude feedback on the amount of anesthetic injected; no feedback for the elapsed time of injection; does not allow the practitioner to speed-up the injection rate.

One device, described in U.S. Pat. No. 5,690,618, addressed some of these issues, but exhibited other problems: very complex to use, requiring the clinician to program various rates and times for each injection; and, no feedback for the elapsed time or cumulative volume of injection.

### DISCLOSURE OF THE INVENTION

It is therefore, an object of the present invention to provide syringe useful in dispensing fluid materials.

It is another object of the invention to provide such a syringe particularly suited for the dispensing of dental materials.

It is a further object of the invention to provide such a syringe that is computer controlled.

It is yet another object of the invention to provide such a syringe with an improved harpoon connector between a drive shaft and a carpule seal.

These and other objects of the invention that will become apparent from the following discussion are carried out by the invention as hereinafter described and claimed.

In general, an electrically controlled syringe for dispensing a fluid material, comprises a power drive unit electrically and operatively connected to a syringe unit via at least one connecting conduit; said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle; a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal; an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit; said harpoon being fabricated from a hard, corrosion resistant, sterilizable material; a stripper ring positioned within said syringe unit and proximate to said carpule plunger seal when said carpule holder is in place upon said syringe unit, such that when said carpule plunger seal is moved from the dispensing to the retracted position, said stripper ring physically engages said carpule plunger seal, preventing further retracting movement of said carpule plunger seal and hence, allowing extraction of said harpoon from said carpule plunger seal; said stripper ring having an inside diameter larger than said harpoon such that said harpoon is receivable therein; and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

In another embodiment of the invention, an electrically controlled syringe for dispensing a fluid material comprises a power drive unit electrically and operatively connected to a syringe unit via at least one connecting conduit; said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle; a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal; an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit; said harpoon being fabricated from a hard, corrosion resistant, sterilizable material; and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

A computer controlled syringe for dispensing a fluid material also comprises a power drive unit electrically and operatively connected to a syringe unit via at least one connecting conduit; said power drive unit including a logic control circuit; said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle; a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal; an electric drive motor operatively affixed to said drive shaft, and operatively connected to said power drive unit, such that the rate of and direction of the longitudinal displacement of said drive shaft is selectable by electrical, operative signals received from said logic control circuit of said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit; said harpoon being fabricated from a hard, corrosion resistant, sterilizable material; and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

A programmable, electrically controlled syringe for dispensing a fluid material according to the invention comprises a power drive unit electrically and operatively connected to a syringe unit via at least one connecting conduit; said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle; a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal; an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a pre-selected longitudinal direction at a pre-selected rate, thereby laterally displacing said operatively connected harpoon and carpule plunger seal, said drive motor being operable with electric power received from said power drive unit; said harpoon being fabricated from a hard, corrosion resistant, sterilizable material; and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

Also according to the invention, an electrically controlled syringe for dispensing a fluid material comprises a power drive unit electrically and operatively connected to a syringe unit via at least one connecting conduit; said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule is caused to flow through said dispensing needle; a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal; an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit; said harpoon being fabricated from a hard, corrosion resistant, sterilizable material; said power drive unit having image displays to provide digital or analog indicia of system parameters selected from the group consisting of elapsed time of dispensing, rate of dispensing, volume of material dispensed, dispensing or aspirating mode, or combinations thereof.

Still another embodiment of the invention comprises an electrically controlled syringe for dispensing a fluid material having a power drive unit electrically and operatively connected to a syringe unit via at least one connecting conduit; said syringe unit having a releasably connected carpule holder, said carpule holder being initially loaded with the material to be dispensed, said carpule holder being releasably connected at one end to said syringe unit and threadably and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle; a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal; an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit; said harpoon being fabricated from a hard, corrosion resistant, sterilizable material; a stripper ring positioned within said syringe unit and proximate to said carpule plunger seal when said carpule holder is in place upon said syringe unit, such that when said carpule plunger seal is moved from the dispensing to the retracted position, said stripper ring physically engages said carpule plunger seal, preventing further retracting movement of said carpule plunger seal and hence, allowing extraction of said harpoon from said carpule plunger seal; said stripper ring having an inside diameter larger than said harpoon such that said harpoon is receivable therein; and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

### BRIEF DISCUSSION OF THE DRAWINGS

FIG. 1 is a perspective view of the harpoon portion of a dental syringe, according to the invention.

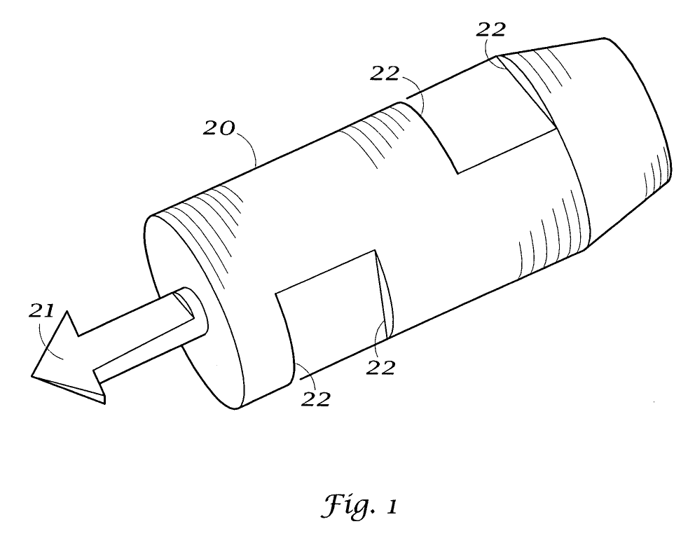

FIG. 2 is a view of the opposite side of the harpoon as shown in FIG. 1.

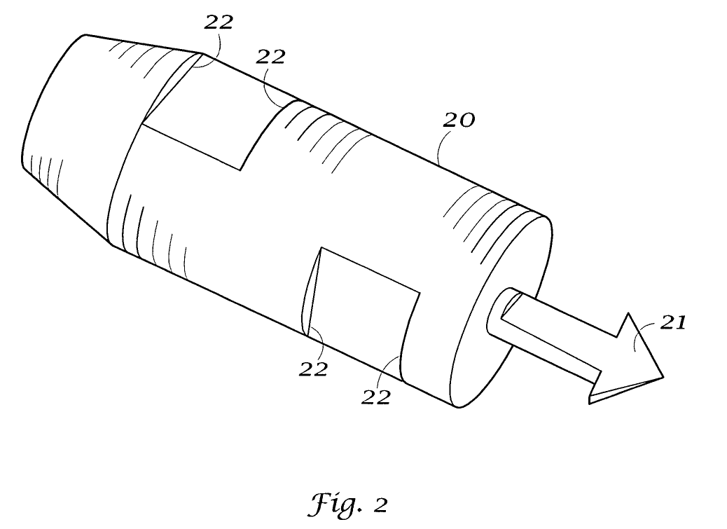

FIG. 3 is a side elevational view of the harpoon shown in FIG. 1.

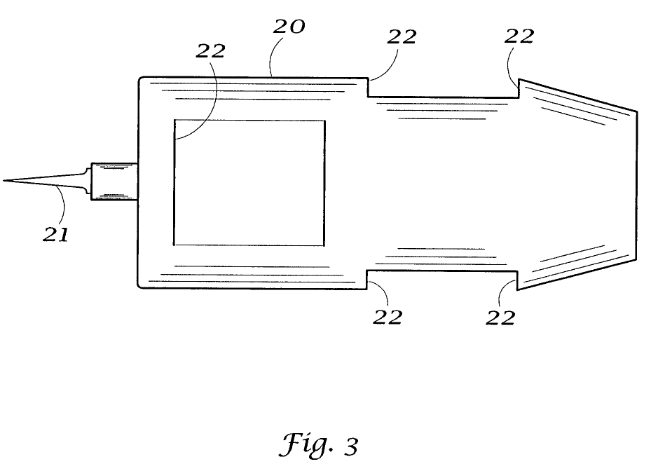

FIG. 4 is a perspective view of a dental syringe according to the present invention.

FIG. 5 is a lengthwise cross-sectional view of the syringe of FIG. 4.

FIG. 6 is another cross-sectional view as in FIG. 5.

FIG. 7 is a closeup view of one portion of the cross-section of FIG. 5, showing the harpoon of FIGS. 1-3 in place in the syringe.

FIG. 8 is a partially schematic representation of a control panel for the computer-controlled syringe according to the present invention.

FIG. 9 is an exploded, perspective view of a portion of the syringe of FIG. 4.

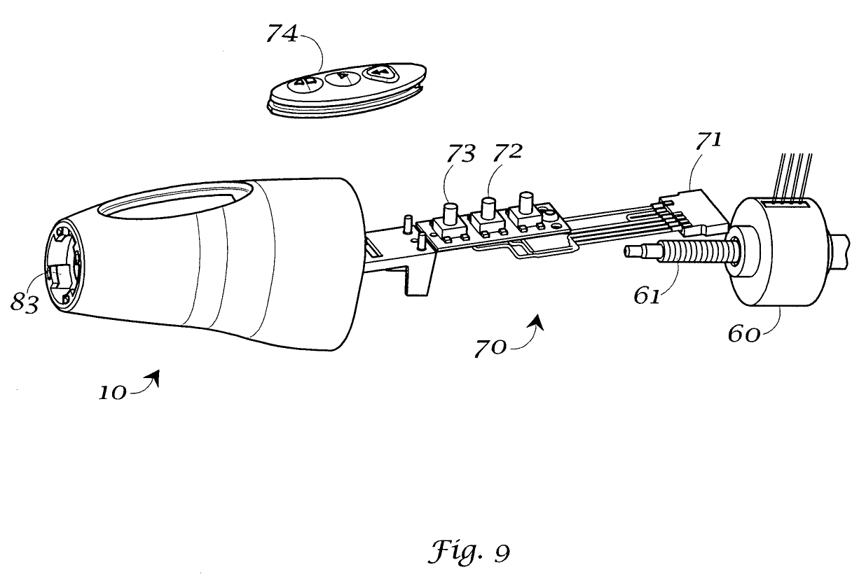

### PREFERRED EMBODIMENTS FOR CARRYING OUT THE INVENTION

An exemplary computer controlled syringe, embodying the concepts of the present invention, is generally shown by the number 10 on the attached drawings. Syringe 10 has a dispensing tip 11 fluidly affixed to a carpule holder 12, which carpule holder 12 is releasably affixed or connected to a syringe power unit 13.

Carpule holder 12 is initially (that is, prior to dispensing) loaded with the material to be dispensed (not shown) by any conventional means, such as a conventional carpule or the like. Any carpule capable of being dispensed by the action of a physically engaging plunger (to be discussed below) is within the scope of the invention. Carpule holder 12 may be affixed to syringe 10 by any conventional means, including for example, bayonet connector 50 at one end of carpule holder 12. At it other end, carpule holder 12 is preferably provided with means to affix or removably affix the dispensing tip 11. In the case of the use of syringe 10 to dispense a dental anesthetic or the like, dispensing tip 11 is a hypodermic needle, which is affixed by conventional means, such as friction, screw threads or the like, to carpule holder 12. Preferably, dispensing tip 11 is fluidly affixed to carpule holder 12, so as to fluidly communicate with the interior thereof, or whatever carpule or the like is employed.

Carpule holder 12 is provided with a carpule plunger seal 32, which is preferably rubber-like in manufacture, for reasons to be discussed. Carpule plunger seal 32 is preferably laterally displaceable within carpule holder 12 to thereby provide for dispensing of material from carpule holder 12 or aspiration of external material through dispensing tip 11. Thus, preferably, carpule plunger seal 32 is selectively, laterally displaceable between a dispensing and a retracting movement. When carpule plunger seal 32 is caused to move toward affixed dispensing tip 11, material in carpule holder 12 is caused to flow toward dispensing tip 11, and when expressed therethrough, is said to have dispensed the material.

Syringe power unit 13 of syringe 10 is preferably provided with an electric drive motor 60, which is employed to laterally displace a drive shaft 61. Motor 60 may be of any conventional design, but is preferably an electrically powered stepper motor with integral internal rotating nut that drives a lead-screw to provide open-loop linear motion. Such motors are commercially available for example, from Haydon Switch and Signal, as well as others. Further, motor 60 should be capable of being controlled as to start and stop of motion, as well as amount of and speed of the lateral displacement of the drive shaft 61, by signals received from an operator, and more preferably received via conduit 41 from power drive unit 40. Motor 60 may also be powered by electricity received through conduit 41 or by any other conventional means, such as batteries located in syringe 10 (not shown). Drive shaft 61 is operatively and releasably connected to carpule plunger seal 32, by any means but preferably by the means described herein.

Drive shaft 61 preferably has an end proximal to and an end distal to carpule holder 12 when carpule holder 12 is connected to syringe 10. In order to effect the connection between drive shaft 61 and carpule plunger seal 32, it is preferred to employ an inventive harpoon 20 that will be hereinafter described.

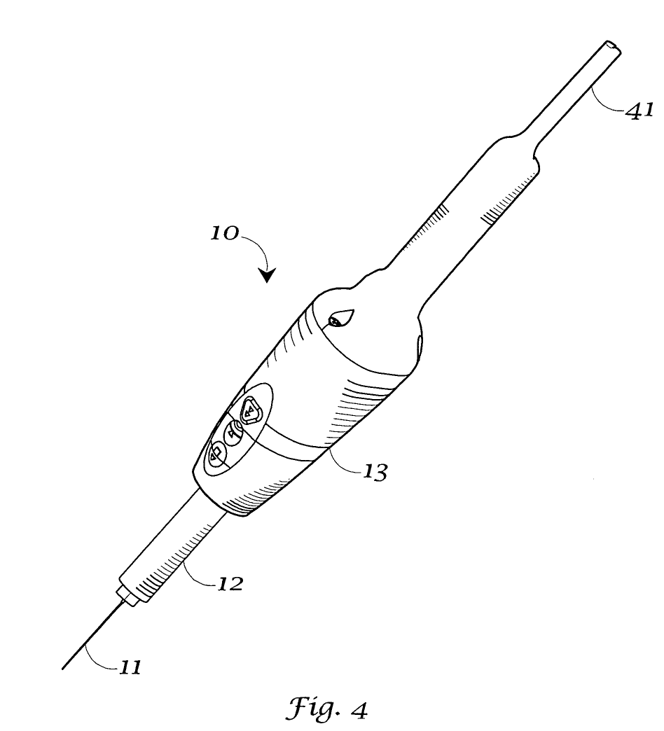

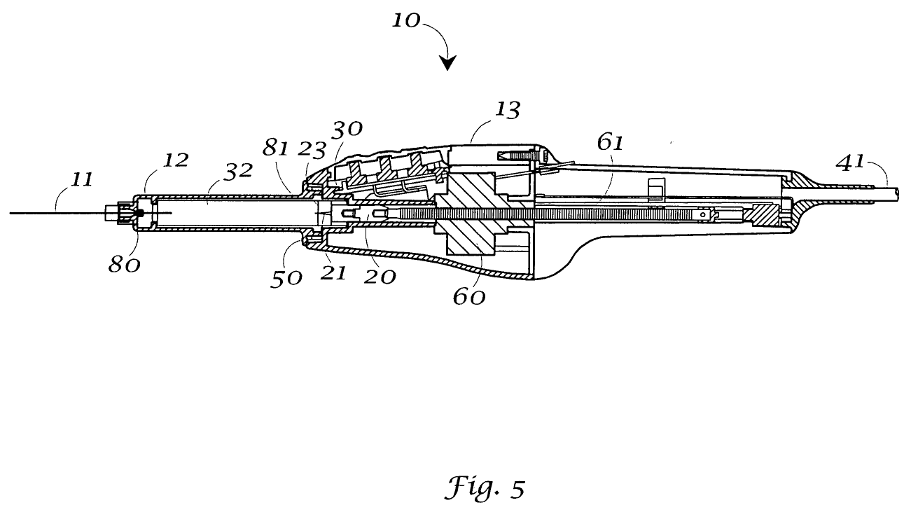

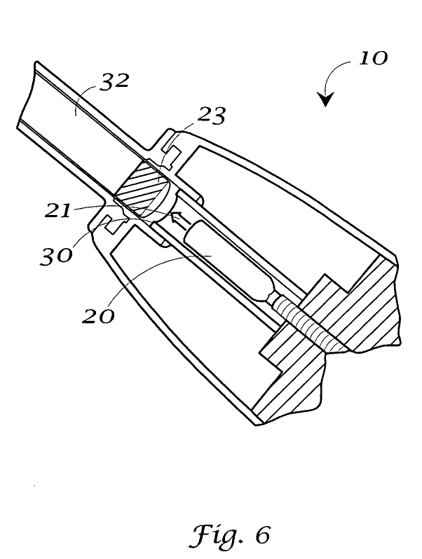

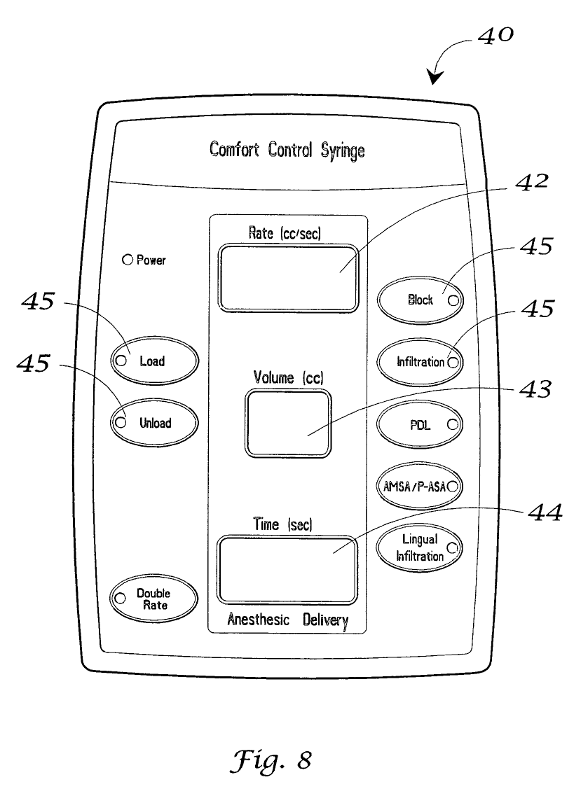

Syringe 10 is operatively and electrically connected to a power drive or base unit 40 (FIG. 8) via a connecting conduit 41 (FIGS. 4-6). Power drive unit 40 via conduit 41 provides electrical signal to syringe 10 to control the operation thereof, and preferably includes a logic control circuit (not shown) of any suitable sort to provide such selected or predetermined control signals. For example, power drive unit 40 may be used to control the flow rate, flow duration, start, stop, elapsed time, volume of dispensed material, direction of material flow, connection to a carpule (known as loading) or disconnecting therefrom (unloading) or the like. The mechanism of such controls will be described below in greater detail. Control signals from power drive unit 40 may be digital or analog, and may be displayed by any suitable means, including using digital readouts 42 (rate of dispensing), 43, (volume of material dispensed), 44 (time of dispensing), or any other desired parameter without limitation. Control mechanisms include buttons 45 for controlling dispensing conditions or parameters, or the like. Power drive unit 40 may be preset for automatic control of dispensing parameters, or such parameters may be individually controlled. As an example of a preset parameter, a button 45 may provide for a doubling of the rate of dispensing of material. By using a logic control circuit or computer, the number of, type of, rate of or the li9ke of all syringe parameters can be preselected, and hence, the device is programmable.

Conduit 41 may also be used to provide electrical power to syringe 10 for purposes to be more fully explained in the following discussion. If required, multiple conduits (not shown) similar to conduit 41 or of some other conventional design, may be employed.

Syringe 10 will be exemplified herein with respect to the dispensing of a dental anesthetic material, it being understood that the invention has application to many materials including the dispensing of medical, industrial or other dental materials.

The general operation of syringe 10 in delivering anesthetics to a patient, is well known in the art, except as otherwise described, noted and claimed. For example, a power driven syringe is shown in U.S. Pat. No. 5,690,618, which is hereby incorporated by reference for such disclosure.

As stated above, an inventive harpoon 20 is provided according to the present invention. Harpoon 20 would typically and preferably be made of stainless steel or other hard, corrosion resistant, sterilizable, material. Harpoon 20 has a unique barb 21 geometry and hardened, knife-like, edges 22 located along its length, with the following advantages: the thin configuration, sharp point, and hard knife edges 22 of the harpoon uniquely allow consistent penetration into all types of elastomer carpule seals, such as carpule plunger seal end 23, even those of hard rubber, with lower force than other gripper types. Thus, it does not require the operator to jam the carpule into the syringe. The swept-back barbs 21 allow the harpoon to consistently remain in the carpule plunger, up to five times more effectively than other gripper types. Thus, aspiration is consistent. This design is easily fabricated at low cost, and has a long life (number of insertions into rubber). The design reduces or eliminates the drawbacks of the vacuum type.

Because harpoon 20 is affixed to carpule plunger seal 32 and to drive shaft 61, lateral displacement of drive shaft 61 also causes lateral displacement of harpoon 20 and hence, also of affixed carpule plunger seal 32.

Another inventive feature of the invention is the incorporation of a stripper ring 30 in the syringe device 10. This ring has an inside diameter (ID) slightly larger than the harpoon 20 but smaller than a standard carpule plunger seal 23. Thus, when the syringe plunger seal 32 is fully retracted, the harpoon 20 is pulled out of the carpule seal 23 while the carpule seal 23 remains within the carpule holder 12, by physical contact between stripper ring 30 and carpule plunger seal 32, thereby preventing further movement of carpule plunger seal 32. Continued retracting movement of drive shaft 61 and affixed harpoon 20 past the point physical contact between stripper ring 30 and carpule plunger seal 32 will cause harpoon 20 to be physically disengaged from carpule plunger seal 32. By being a ring of proper dimension, harpoon 20 and drive shaft 61 maybe received within stripper ring 30.

Harpoon 20 has the following advantages: it readily penetrates the carpule plunger seal 32 with only moderate force, even plungers made of high durometer rubber; it does not pull out of the carpule plunger seal 32, even in repeated aspirations; nonetheless, it can be readily stripped from the carpule plunger seal 32 with proper syringe design, using stripper ring 30; its shape is suited to low-cost manufacturing.

As discussed above, it is preferred to control dispensing parameters of syringe 10 by control signals or commands received from power drive unit 40. It is also an advantage of the present invention, that an operator can control some or all such parameters by use of a secondary injection control mechanism 70 (FIG. 11) located proximate to syringe 10 and distal to power drive unit 41. Secondary injection control mechanism 70 is operatively connected to said drive motor 60, such as by connector 71, to control the longitudinal displacement direction or rate of drive shaft 61, by command signals that start, stop, adjust speed, torque, or the like of motor 60, and hence, the injection rate of the material dispensed from dispensing tip 11. Secondary injection control mechanism 70 may be of any design such as a switchboard 72, button 73 design as depicted in the drawings. For anesthetic purposes, secondary control mechanism 70 may be covered with for example, cap 74.

The syringe 10 according to the present invention also divides the delivery of anesthesia into two phases. According to the method of the present invention, during the first 10 seconds of the injection, anesthetic is delivered at an extremely slow rate to maximize patient comfort. The injection rate then automatically increases to the preprogrammed rate associated with the injection type you have selected. The following steps refer to the control panel of FIG. 8.

A. Select your injection by depressing the appropriate button under Injection Technique. The injection rate will be displayed in the box title Rate cc/sec.

B. Once the injection technique is selected, you are ready to proceed.

C. Aspiration is achieved by pressing and releasing the middle button on the handpiece, once.

D. After aspiration, press an release the front start/stop button on the handpiece to initiate the injection.

E. At any time during the injection you may stop by simply pressing the front start/stop button on the handpiece to stop the program.

F. You can double the rate of injection at any time by pressing the back button on the handpiece or the double rate button on the base unit. To turn this feature off, simply push the Double button again on either the handpiece or the control box.

G. When you are through with the injection, press and release the front start/stop button once to stop the program.

H. If you inject into a new site, change your injection technique setting if necessary, follow steps A-D and the program will automatically start over.

I. When finished, re-sheath the needle and set the handpiece in the holder.”

Other physical embodiments utilizing the same simplified control scheme. For example, a unitary battery operated handpiece.

It will be appreciated that the syringe according to the present invention is simple to use. Control choices are directly related to known clinical practice rather than arcane rates and times. It provides clinically useful display information.

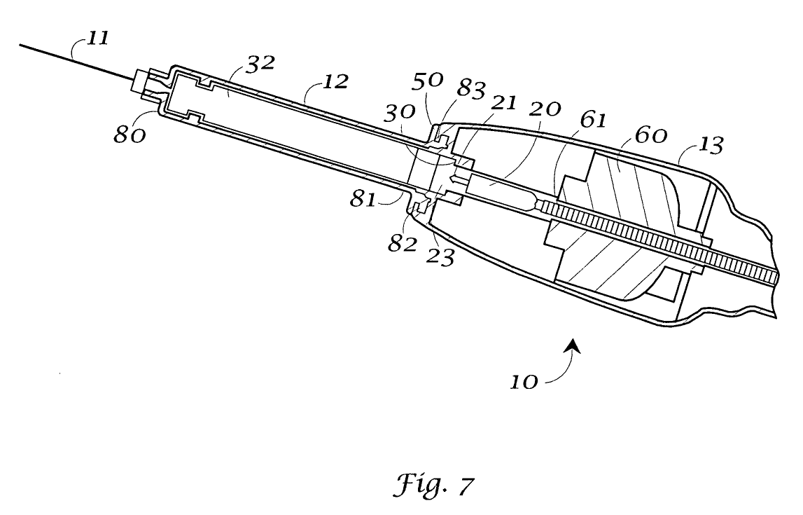

As shown in FIG. 7, carpule holder 12 is provided with a discharge end 80 and a connector end 81. Connector end 81 is used to removably affix carpule holder 12 to syringe 10. Connector end 81 is provided with wedge lugs 82, which physically engage circumferential lips 83 carried by syringe 10.

It should therefore be apparent that the dental syringe as described herein carries out the object of the invention and otherwise provides an advance and contribution to the art. The invention has been exemplified with respect to drawings and description, without an attempt to provide a depiction or description of every embodiment of the event of device or method. Those skilled in the art will readily understand that various sizes, components and method steps can be employed and still fall within the scope of the present invention.

What is claimed is:

1. An electrically controlled syringe for dispensing a fluid material, comprising:
a power drive unit electrically and operatively connected to a syringe unit via at least one connecting conduit;
said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle;
a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal;
an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit;
said harpoon being fabricated from a hard, corrosion resistant, sterilizable material;
a stripper ring positioned within said syringe unit and proximate to said carpule plunger seal when said carpule holder is in place upon said syringe unit, such that when said carpule plunger seal is moved from the dispensing to the retracted position, said stripper ring physically engages said carpule plunger seal, preventing further retracting movement of said carpule plunger seal and hence, allowing extraction of said harpoon from said carpule plunger seal; said stripper ring having an inside diameter larger than said harpoon such that said harpoon is receivable therein;
and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

2. An electrically controlled syringe for dispensing a fluid material, comprising:
a power drive unit electrically and operatively connected to a syringe unit via at least one connecting conduit;
said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle;
a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal;
an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit;
said harpoon being fabricated from a hard, corrosion resistant, sterilizable material;
and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

3. A computer controlled syringe for dispensing a fluid material, comprising:
a power drive unit electrically and operatively connected to a syringe unit via at least one connecting conduit; said power drive unit including a logic control circuit;
said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle;
a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal;
an electric drive motor operatively affixed to said drive shaft, and operatively connected to said power drive unit, such that the rate of and direction of the longitudinal displacement of said drive shaft is selectable by electrical, operative signals received from said logic control circuit of said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit;
said harpoon being fabricated from a hard, corrosion resistant, sterilizable material;
and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

4. A programmable, electrically controlled syringe for dispensing a fluid material, comprising:
a power drive unit electrically and operatively connected to a syringe unit via at least one connecting conduit;
said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle;
a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal;
an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a pre-selected longitudinal direction at a pre-selected rate, thereby laterally displacing said operatively connected harpoon and carpule plunger seal, said drive motor being operable with electric power received from said power drive unit;
said harpoon being fabricated from a hard, corrosion resistant, sterilizable material;
and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.

5. An electrically controlled syringe for dispensing a fluid material, comprising:
a power drive unit electrically and operatively connected to a syringe unit via at least one connecting conduit;
said syringe unit having a releasably connected carpule holder, said carpule holder being initially loaded with the material to be dispensed, said carpule holder being releasably connected at one end to said syringe unit and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle;
a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges positioned along its length, such that said barbed point is insertable into said rubber-like carpule plunger seal, and is selectively prevented from being removed by physical contact between said barbed point and said carpule plunger seal;
an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit;
said harpoon being fabricated from a hard, corrosion resistant, sterilizable material;
said power drive unit having image displays to provide digital or analog indicia of system parameters selected from the group consisting of elapsed time of dispensing, rate of dispensing, volume of material dispensed, dispensing or aspirating mode, or combinations thereof.

6. An electrically controlled syringe for dispensing a fluid material, comprising:
a power drive unit electrically and operatively connected to a syringe unit via at least one connecting conduit;
said syringe unit having a releasably connected carpule holder, said carpule holder being initially loaded with the material to be dispensed, said carpule holder being releasably connected at one end to said syringe unit and threadably and fluidly connected at its other end to a dispensing needle, said carpule holder having a rubber-like plunger seal laterally displaceable therein between a dispensing and a retracted position, such that when said carpule plunger seal is moved toward a dispensing position, the material in said carpule holder is caused to flow through said dispensing needle;
a longitudinally movable, powered drive shaft in said syringe unit and releasably connected to said carpule plunger seal, said drive shaft having an end proximal to and an end distal to said carpule plunger seal when said carpule holder is in place on said syringe unit; wherein said drive shaft is provided with a harpoon at said proximal end; said harpoon having a swept-back, barbed point and knife edges along its length, such that said barbed point is insertable into said rubber-like carpule plunger seal, and is selectively prevented from being removed by physical contact between said barbed point and said carpule plunger seal;
an electric drive motor operatively affixed to said drive shaft, and operatively connected to said power drive unit, such that the rate of and direction of the longitudinal displacement of said drive shaft is selectable by electrical, operative signals received from said power drive unit to cause said drive motor to displace said drive shaft in a selected longitudinal direction at a selected rate, thereby laterally displacing said operatively connected harpoon and carpule plunger seal, said drive motor being operable with electric power received from said power drive unit;
said harpoon being fabricated from a hard, corrosion resistant, sterilizable material;
a stripper ring positioned within said syringe unit and proximate to said carpule plunger seal when said carpule holder is in place upon said syringe unit, such that when said carpule plunger seal is moved from the dispensing to the retracted position, said stripper ring physically engages said carpule plunger seal, preventing further retracting movement of said carpule plunger seal and hence, allowing extraction of said harpoon from said carpule plunger seal; said stripper ring having an inside diameter larger than said harpoon such that said harpoon is receivable therein;
and at least one secondary injection control mechanism located proximate to said syringe unit and distal to said power drive unit, wherein said secondary injection control mechanism is operatively connected to said syringe drive motor to control the longitudinal displacement direction or rate of said drive shaft, and hence, the injection rate of the material dispensed from said needle.
